(19) United States
(12) Patent Application Publication
(10) Pub. No.: US 2008/0234317 A1
(43) Pub. Date: Sep. 25, 2008

(54) METHODS FOR THE IMPROVEMENT OF MEMORY AND NEUROLOGICAL FUNCTION COMPRISING THE ADMINISTRATION OF COMPOSITIONS THAT ACT AS INHIBITORS OF THE SODIUM PROTON (NA+ /H+ ) EXCHANGER, SUBTYPE 5 (NHE-5)

(75) Inventors: Heinz-Werner KLEEMANN, Frankfurt am Main (DE); Hans-Jochen LANG, Hofheim (DE); Uwe HEINELT, Frankfurt am Main (DE); Armin HOFMEISTER, Frankfurt am Main (DE); Holger GAUL, Frankfurt am Main (DE); Ulrich Hendrich SCHROEDER, Eussenheim (DE); Klaus REYMANN, Niederndodeleben (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/046,920

(22) Filed: Mar. 12, 2008

Related U.S. Application Data
(63) Continuation of application No. PCT/EP2006/008771, filed on Sep. 8, 2006.

(30) Foreign Application Priority Data
Sep. 20, 2005 (DE) 102005044815.1

(51) Int. Cl. A61K 31/47; A61P 25/00

(52) U.S. Cl. 514/307

(57) ABSTRACT

The present invention comprises the use of pharmaceutical compositions that are effective in the inhibition of the Na⁺/H⁺ exchanger, subtype 5 (NHE-5) as inhibitors of cellular NHE-5 enhance long term potentiation (LTP) and are therefore effective in the treatment of memory impairments, dementing disorders, and for improving memory. Particularly suitable for the treatment of neurodegenerative disorders, memory impairments and dementing disorders, and for improving memory, are the following NHE-5 inhibitors of the formula I

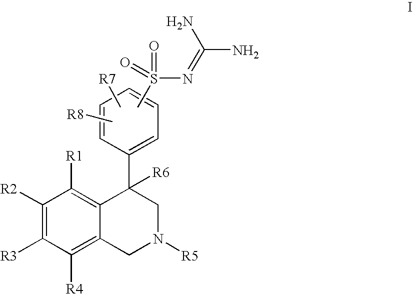

Wherein the R1-R8 substituents are further defined herein:

### CROSS-REFERENCE TO RELATED APPLICATIONS

This application is a continuation application of PCT/EP2006/008771 filed on Sep. 8, 2006 which claims priority from German application Ser. No. 10/2005044815.1 filed on Sep. 20, 2005.

### FIELD OF THE INVENTION

The present invention relates generally to pharmaceutical compositions useful in the treatment of neurodegenerative and psychiatric disorders and methods comprising their administration for the treatment thereof. More particularly, the present invention comprises the use of pharmaceutical compositions that are effective in the inhibition of the Na⁺/H⁺ exchanger, subtype 5 (NHE-5) as inhibitors of cellular NHE-5 enhance long term potentiation (LTP) and are therefore effective in the treatment of memory impairments, dementing disorders, and for improving memory.

### BACKGROUND OF THE INVENTION

The term dementia refers to a decline in intellectual capacity. It is understood to mean in particular the decrease in memory and thinking ability. Dementia in the elderly or “senile dementia” refers to a progressive, acquired intellectual decline in people of advanced age which is attributable to structural and/or metabolic abnormalities in the central nervous system. Approximately 7% of the population over 65 years of age suffers from dementia of varying severity. The causes of dementia vary: Alzheimer's disease is the most common form, accounting for up to 50% of the dementia present in the elderly followed by vascular dementias such as multi-infarct dementia, and combinations of these two forms. Much rarer causes are tau mutations, prion diseases, polyglutamine expansion disorders such as Huntington's chorea and spinocerebellar ataxias, and Parkinsonism. Also known in addition to these are secondary dementias following and/or associated with infections (e.g. with HIV), brain traumas, brain tumors or intoxications (e.g. with alcohol).

The concept of memory consolidation is based on the ability of new memories to stabilize over the course of time and thus become less sensitive to interference by new information and dysfunctions of the brain. It is possible with the aid of the prevailing cellular model of long-term potentiation (LTP) to investigate essential aspects and mechanisms of memory formation and consolidation (Neuroscientist. 9: 463-474.2003; Brain Res Brain Res Rev. 45: 30-37, 2004; Physiol Rev. 84: 87-136, 2004).

One of the most important regions of the brain in which information is stored and processed is the hippocampus formation. It has long been known that certain patterns of electrical stimulation (tetanization) in the hippocampus lead to changes in synaptic efficiency (Bliss and Lomo, J Physiol. 232: 331-356, 1973) which are now referred to as ‘long-term potentiation’ or ‘LTP’, and which have subsequently been confirmed in other areas of the brain in a wide variety of mammals, both in vitro and in vivo. LTP is now regarded as an important component of the neuronal mechanism underlying learning and memory. It is further known that a weak LTP correlates with short-term memory, and a strong LTP with long-term memory (J Neurosci. 20: 7631-7639, 2000; Proc Natl Acad Sci USA. 97: 8116-8121, 2000).

The hippocampus plays a central role in episodic, spatial and declarative learning and memory processes, it is essential for spatial orientation and recall of spatial structures and plays an important role in the control of autonomic and vegetative functions (McEwen 1999, Stress and hippocampal plasticity, Annual Review of Neuroscience 22: 105-122). In human dementing disorders there is usually impairment of learning and memory processes in which the hippocampus is involved. Animal experiments on other mammals have shown similar results. Thus, it was possible to show that aged mice have deficits in spatial memory and in the LTP compared with young mice, and that substances which improved the LTP simultaneously reduced the memory deficits (Bach et al. 1999, Age-related defects in spatial memory are correlated with defects in the late phase of hippocampal long-term potentiation in vitro and are attenuated by drugs that enhance the cAMP signaling pathway. Proc Natl Acad Sci USA. 27; 96:5280-5; Fujii & Sumikawa 2001, Acute and chronic nicotine exposure reverse age-related declines in the induction of long-term potentiation in the rat hippocampus. Brain Res. 894:347-53, Clayton et al. 2002, A hippocampal NR2B deficit can mimic age-related changes in long-term potentiation and spatial learning in the Fischer 344 rat. J Neurosci. 22:3628-37).

It was possible to show in vivo and in vitro on transgenic animals and by administration of beta-amyloid peptides that the peptides adversely affect LTP or interfere with maintenance thereof (Ye & Qiao 1999, Suppressive action produced by beta-amyloid peptide fragment 31-35 on long-term potentiation in rat hippocampus is N-methyl-D-aspartate receptor-independent: it's offset by (−)huperzine A. Neurosci Lett. 275:187-90. Rowan et al 2003, Synaptic plasticity in animal models of early Alzheimer's disease. Philos Trans R Soc Lond B Biol Sci. 358: 821-8, Gureviciene et al. 2004, Normal induction but accelerated decay of LTP in APP+PS1 transgenic mice. Neurobiol Dis 15:188-95). It was possible to correct the impairment of the LTP and of memory functions by rolipram and cholinesterase inhibitors like those also employed in human Alzheimer's therapy (Gong et al. 2004, Persistent improvement in synaptic and cognitive functions in an Alzheimer mouse model after rolipram treatment. J Clin Invest. 114:1624-34.)

It is thus to be expected that substances which improve the LTP will also have a beneficial effect on disorders associated with cognitive impairments and dementia.

It has surprisingly been found that inhibitors of cellular NHE-5 enhance LTP. A memory-improving effect of the inhibitor in dementing disorders such as Alzheimer's and Alzheimer-like forms of dementia is therefore to be expected. The use of an NHE-5 inhibitor has the advantage over the active ingredients employed to date for these disorders, such as acetylcholinesterase inhibitors, that systemic effects are expected to be slight or absent, because NHE-5 is expressed only in neurons and is therefore brain-specific (Am. J. Physiol. Cell. Physiol. 281: C1146-C1157, 2001). NHE-5 inhibitors are therefore suitable for the treatment of neurodegenerative disorders, memory impairments and dementing disorders such as dementia in the elderly, Alzheimer's, vascular dementias such as, for example, multi-infarct dementia, combinations of Alzheimer's and cerebrovascular disorders, tau mutations, prion diseases, polyglutamine expansion disorders such as, for example, Huntington's chorea and spinocerebellar ataxias, and Parkinsonism, and for improving memory. NHE-5 inhibitors are further suitable for the treatment of secondary dementias following and/or associated with infections such as, for example, with HIV, brain traumas, brain tumors or intoxications such as, for example, with alcohol.

### SUMMARY OF THE INVENTION

The present invention comprises the use of pharmaceutical compositions that are effective in the inhibition of the Na⁺/H⁺ exchanger, subtype 5 (NHE-5) as inhibitors of cellular NHE-5 enhance long term potentiation (LTP) and are therefore effective in the treatment of memory impairments, dementing disorders, and for improving memory. Particularly suitable for the treatment of neurodegenerative disorders, memory impairments and dementing disorders, and for improving memory, are the following NHE-5 inhibitors of the formula I

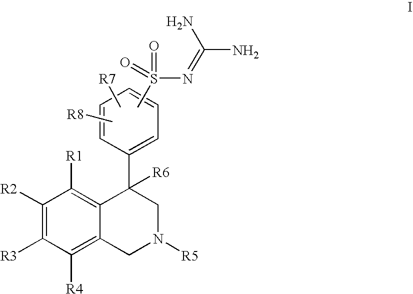

Wherein the R1-R8 substituents are further defined herein:

### DETAILED DESCRIPTION OF THE INVENTION

The present invention comprises the use of pharmaceutical compositions that are effective in the inhibition of the Na⁺/H⁺ exchanger, subtype 5 (NHE-5) as inhibitors of cellular NHE-5 enhance long term potentiation (LTP) and are therefore effective in the treatment of memory impairments, dementing disorders, and for improving memory. Particularly suitable for the treatment of neurodegenerative disorders, memory impairments and dementing disorders, and for improving memory, are the following NHE-5 inhibitors of the formula I

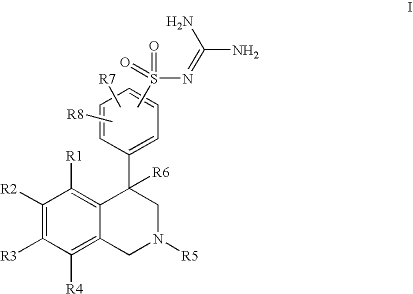

wherein:
R1, R2, R3 and R4 are independently selected from the group consisting of hydrogen, F, Cl, Br, I, CN, NO₂ and R11-(CₘH₂ₘ)-Aₙ, wherein
   m is zero, 1, 2, 3 or 4;
   n is zero or 1;
   R11 is hydrogen, methyl or CₚF₂ₚ₊₁;
   A is oxygen, NH, N(CH₃) or S(O)_{q};
   p is 1, 2 or 3;
   q is zero, 1 or 2;
R5 is selected from the group consisting of hydrogen, alkyl having 1, 2, 3, 4, 5 or 6 carbon atoms or cycloalkyl having 3, 4, 5 or 6 Carbon atoms;
R6 is selected from the group consisting of hydrogen, OH, F, CF₃, alkyl having 1, 2, 3 or 4 Carbon atoms or cycloalkyl having 3, 4, 5 or 6 Carbon atoms;
R7 and R8 are independently selected from the group consisting of hydrogen, F, Cl, Br, CN, CO₂R12, NR13R14 or R16-(CₘₘH₂ₘₘ)-Eₙₙ-; wherein
R12 is selected from the group consisting of hydrogen, alkyl having 1, 2, 3 or 4 carbon atoms or cycloalkyl having 3, 4, 5 or 6 carbon atoms;
R13 and R14 are independently selected from the group consisting of hydrogen, alkyl having 1, 2, 3 or 4 carbon atoms or cycloalkyl having 3, 4, 5 or 6 carbon atoms;
   or
R13 and R14 together with the nitrogen atom to which they are bonded form a 4, 5, 6 or 7 membered ring in which one CH₂ group may be replaced by NR15, S or oxygen; wherein:
R15 is selected from the group consisting of hydrogen, an alkyl having 1, 2, 3 or 4 carbon atoms or a cycloalkyl having 3, 4, 5 or 6 carbon atoms; and
   mm is zero, 1, 2, 3 or 4;
   nn is zero or 1;
   R16 is hydrogen, methyl or CₚₚF₂ₚₚ₊₁;
   E is oxygen or S(O)_{qq};
   pp is 1, 2 or 3;
   qq is zero, 1 or 2;
      and the pharmaceutically acceptable salts and trifluoroacetates thereof.

Preferably, compounds of formula I comprise those structures in which:
R1, R2, R3 and R4 are independently selected from the group consisting of hydrogen, F, Cl, Br, CN or R11-(CₘH₂ₘ)-Aₙ-;
   m is zero or 1;
   n is zero or 1;
   R11 is hydrogen, methyl or CₚF₂ₚ₊₁;
   A is oxygen, NCH₃ or S(O)_{q};
   p is 1 or 2; and
   q is zero, 1 or 2;
R5 is selected from the group consisting of hydrogen, methyl, ethyl and cyclopropyl;
R6 is hydrogen or methyl;
R7 and R8 are independently selected from the group consisting of hydrogen, F, Cl, CN, CO₂R12, NR13R14 and R16-(CₘₘH₂ₘₘ)-Eₙₙ-;
R12 is hydrogen, methyl or ethyl;
R13 and R14 are independently selected from the group consisting of hydrogen, alkyl having 1, 2, 3 or 4 carbon atoms or cycloalkyl having 3, 4, 5 or 6 carbon atoms;
   or
R13 and R14 together with the nitrogen atom to which they are bonded form a 5, 6 or 7 membered ring in which one CH₂ group may be replaced by NR15, S or oxygen;
R15 is selected from the group consisting of hydrogen, alkyl having 1, 2, 3 or 4 carbon atoms or cycloalkyl having 3, 4, 5 or 6 carbon atoms; and
   Mm is zero, 1 or 2;
   nn is zero or 1;
   R16 is hydrogen, methyl or CₚₚF₂ₚₚ₊₁;
   E is oxygen or S(O)_{qq}; wherein
      pp is 1 or 2;
      qq is zero, 1 or 2;
         and the pharmaceutically acceptable salts and trifluoroacetates thereof.

More preferably, compounds of formula I comprise those structures in which:

R1 and R3 are both hydrogen;
R2 and R4 are independently selected from the group consisting of hydrogen, F, Cl, NH₂, NHCH₃ and N(CH₃)₂;
R5 is hydrogen, methyl, ethyl or cyclopropyl;
R6 is hydrogen or methyl;
R7 and R8 are both hydrogen;
and the pharmaceutically acceptable salts and trifluoroacetates thereof.

Most preferably, the compounds defined by formula I are N-diaminomethylene-4-(6,8-dichloro-2-methyl-1,2,3,4-tetrahydroisoquinolin-4-yl)benzenesulfonamide and its pharmaceutically acceptable salts and trifluoroacetates.

Another preferred group of compounds are those of formula I in which the radicals R1, R2, R3 and R4 are independently selected from the group consisting of hydrogen, F, Cl, Br, CN and R11-(CₘH₂ₘ)-Aₙ-, where m and n are independently selected from the group consisting of zero or 1, R11 is hydrogen, methyl or CₚF₂ₚ₊₁, and A is oxygen, NCH₃ or S(O)_{q}, where p is 1 or 2 and q is zero, 1 or 2; Even more preferably, compounds of the present invention comprise those of formula I in which R1 and R3 are hydrogen, and R2 and R4 are independently selected from the group consisting of hydrogen, F, Cl, NH₂, NHCH₃ and N(CH₃)₂, for example Cl. In this case, most preferably, compounds of formula I are those in which R2 and R4 are not hydrogen.

Preferably, an embodiment of the present invention comprises compounds of formula I in which R5 is selected from the group consisting of hydrogen, methyl, ethyl and cyclopropyl, for example, methyl.

Also preferably, the compounds formula I are those in which in which R6 is hydrogen or methyl.

Yet another preferred embodiment are those compounds of formula I in which R7 and R8 are independently selected from the group consisting of hydrogen, F, Cl, CN, CO₂R12, NR13R14 and R16-(CₘₘH₂ₘₘ)-Eₙₙ-, wherein R12 is hydrogen, methyl or ethyl, R13 and R14 are independently selected from the group consisting of hydrogen, alkyl having 1, 2, 3 or 4 carbon atoms or cycloalkyl having 3, 4, 5 or 6 carbon atoms, or R13 and R14 together with the nitrogen atom to which they are bonded form a 5-, 6- or 7-membered ring in which one CH₂ group may be replaced by NR15, S or oxygen, and where R15 is hydrogen, alkyl having 1, 2, 3 or 4 carbon atoms or cycloalkyl having 3, 4, 5 or 6 Carbon atoms, and where mm is zero, 1 or 2, nn is zero or 1, and R16 is hydrogen, methyl or CₚₚF₂ₚₚ₊₁, where E is oxygen or S(O)_{q}q, where pp is 1 or 2 and qq is zero, 1 or 2; particular preference is given to those compounds of the formula I in which R7 and R8 are hydrogen.

If the compounds of the formula I contain one or more centers of asymmetry, these may independently of one another have both the S and the R configuration. The compounds can be in the form of optical isomers, of diastereomers, of racemates or of mixtures in all ratios thereof.

The present invention encompasses all possible tautomeric forms of the compounds of the formula I.

The present invention also comprises derivatives of the compounds of formula I, for example solvates such as hydrates and alcohol adducts, esters, prodrugs and other physiologically acceptable derivatives of the compounds of the formula I, and active metabolites of the compounds of the formula I. The invention likewise encompasses all crystal modifications of the compounds of the formula I.

Alkyl radicals may be straight-chain or branched. This also applies if they have substituents or occur as substituents of other radicals, for example in fluoroalkyl radicals or alkoxy radicals. Examples of alkyl radicals are methyl, ethyl, n-propyl, isopropyl (=1-methylethyl), n-butyl, isobutyl (=2-methylpropyl), sec-butyl (=1-methylpropyl), tert-butyl (=1,1-dimethylethyl), n-pentyl, isopentyl, tert-pentyl, neopentyl and hexyl. Preferred alkyl radicals are methyl, ethyl, n-propyl, isopropyl and n-butyl. One or more, for example 1, 2, 3, 4, 5, 6, 7, 8, 9, 10, 11, 12, 13 or 14, hydrogen atoms in alkyl radicals may be replaced by fluorine atoms. Examples of such fluoroalkyl radicals are trifluoromethyl, 2,2,2-trifluoroethyl, pentafluoroethyl, heptafluoroisopropyl. Substituted alkyl radicals may be substituted in any positions.

Alkylene radicals such as, for example, CₘH₂ₘ, CₘₘH₂ₘₘ or CᵣH₂ᵣ may be straight-chain or branched. This also applies if they have substituents or occur as substituents of other radicals, for example in fluoroalkylene radicals such as, for example, in CₚF₂ₚ and CₚₚF₂ₚₚ. Examples of alkylene radicals are methylene, ethylene, 1-methylmethylene, propylene, 1-methylethylene, butylene, 1-propyl-methylene, 1-ethyl-1-methylmethylene, 1,2-dimethylethylene, 1,1-dimethylmethylene, 1-ethylethylene, 1-methylpropylene, 2-methylpropylene, pentylene, 1-butylmethylene, 1-propylethylene, 1-methyl-2-ethylethylene, 1,2-dimethylpropylene, 1,3-dimethylpropylene, 2,2-dimethylpropylene, hexylene and 1-methylpentylene. One or more hydrogen atoms in the alkylene radicals may be replaced by fluorine atoms. Substituted alkylene radicals may be substituted in any positions. One or more CH₂ groups in the alkylene radicals may be replaced by oxygen, S, NH, N-alkyl or N-cycloalkyl.

Examples of cycloalkyl radicals are cyclopropyl, cyclobutyl, cyclopentyl or cyclohexyl. One or more hydrogen atoms in the cycloalkyl radicals may be replaced by fluorine atoms. Substituted cycloalkyl radicals may be substituted in any positions. Cycloalkyl radicals may also be in branched form, as alkylcycloalkyl or cycloalkylalkyl, for example methylcyclohexyl or cyclohexylmethyl.

Examples of rings derived from NR13R14, where R13 and R14 form with the nitrogen atom to which they are bonded a 4, 5, 6 or 7 membered ring in which one CH₂ group may be replaced by NR15, S or oxygen, are morpholine, pyrrolidine, piperidine, piperazine and N-methylpiperazine.

If a variable occurs more than once as component, the definitions of the variable are independent of one another for each occurrence.

If the compounds of the formula I comprise one or more acidic or basic groups or one or more basic heterocycles, the corresponding physiologically or toxicologically acceptable salts also belong to the invention, especially the pharmaceutically usable salts. Thus, the compounds of the formula I may be deprotonated on an acidic group and be used for example as alkali metal salts, preferably sodium or potassium salts, or as ammonium salts, for example as salts with ammonia or organic amines or amino acids. Since compounds of the formula I always comprise at least one basic group, they can also be prepared in the form of their physiologically tolerated acid addition salts, e.g. with the following acids: from inorganic acids such as hydrochloric acid, sulfuric acid or phosphoric acid or from organic acids such as acetic acid, citric acid, tartaric acid, lactic acid, malonic acid, methanesulfonic acid, fumaric acid. Suitable acid addition salts in this connection are salts of all pharmacologically acceptable acids, for example halides, in particular hydrochlorides, lactates, sulfates, citrates, tartrates, acetates, phosphates, methylsulfonates, p-toluenesulfonates, adipates, fumarates, gluconates, glutamates, glycerolphosphates, maleates and pamoates (this group also corresponds to the physiologically acceptable anions); but also trifluoroacetates.

The compounds of the formula I described herein can be prepared by chlorosulfonation of compounds of the formula VIII by processes known to the skilled worker with subsequent reaction with guanidine by processes known to the skilled worker (as described for example in Synthetic Communications, 33(7), 1073; 2003).

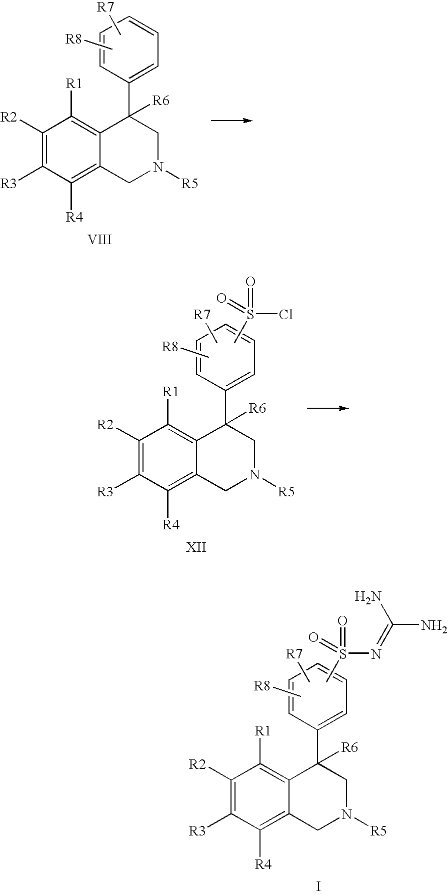

The intermediate of the formula XII obtained after the chlorosulfonation does not need to be isolated, but can be reacted directly further with guanidine.

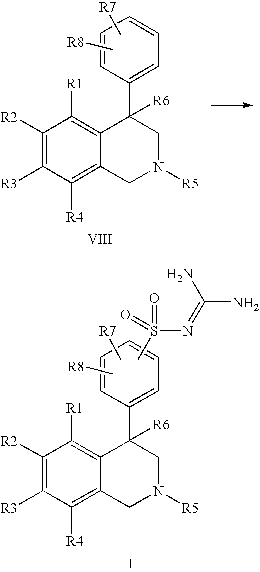

The starting compounds of the formula VIII can be prepared as follows:

It is possible by reducing the carbonyl group in compounds of the formula VI, for example with sodium borohydride, and subsequent acid- or base-catalyzed cyclization of the resulting alcohols of the formula VII (cf. Tetrahedron Lett. 1989, 30, 5837; Org. Prep. Proced. Int. 1995, 27, 513) to prepare tetrahydroisoquinolines of the formula VIIIa by processes known to the skilled worker, where R1 to R8 have the abovementioned meaning.

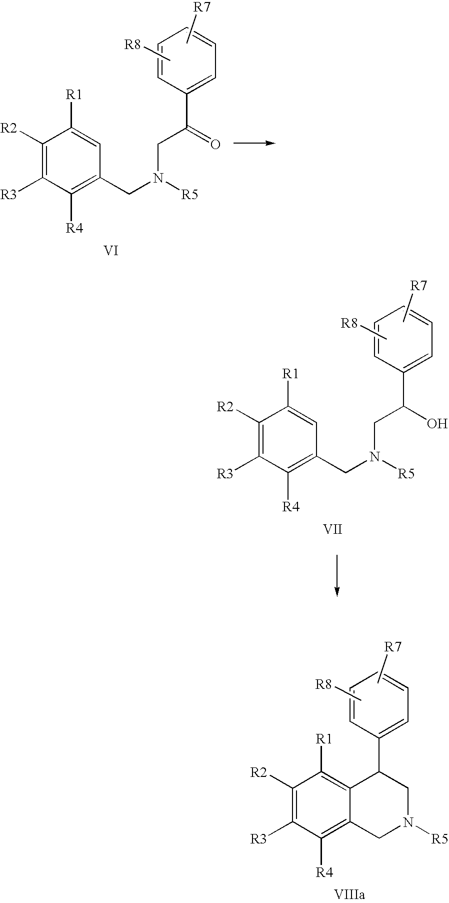

Alkyl-branched compounds of the formula I in which R6 is not hydrogen can be prepared by alkylating the corresponding di-phenylacetic esters of the formula IX in the alpha position with R6 by known methods. The compounds of the formula X can be converted by standard processes until the corresponding amides of the formula XI which are converted in the Pictet-Spengler-analogous reaction into the desired tetrahydroisoquinolines of the formula VIIIb (cf. Tetrahedron 1987, 43, 439; Chem. Pharm. Bull. 1985, 33, 340), where R1 to R8 are as defined above, and LG corresponds to a leaving group conventional for alkylations, such as, for example, bromide, chloride, tosylate or mesylate.

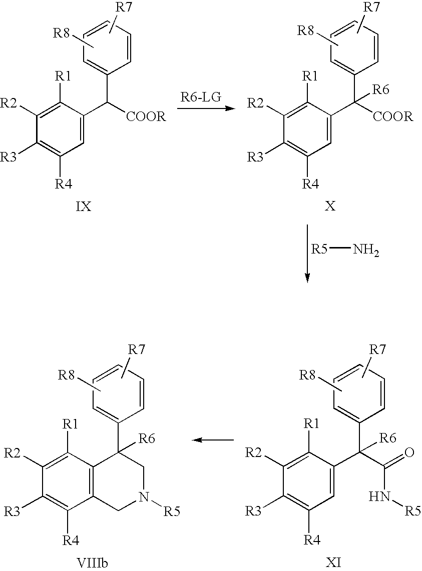

The compounds of formula VI employed above are preferably prepared from benzylamines of the formula IV in a manner known to one skilled in the art and from the appropriate amino-substituted alpha-bromoacetophenone compounds of the formula V, where R1 to R8 are as defined above,

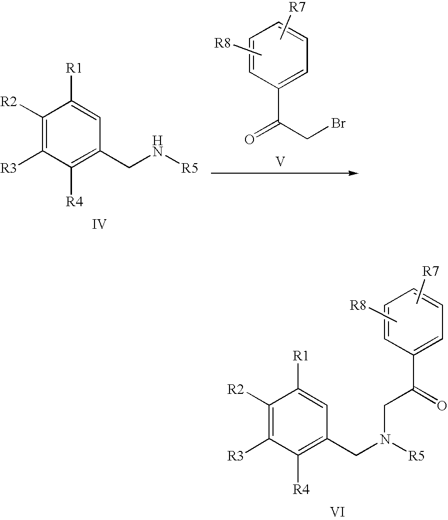

The alpha-bromoacetophenone compounds of the formula V can be obtained in processes known from the literature from the corresponding acetophenone precursors by bromination.

The benzylamine precursors of the formula IV can, if not obtainable by purchase, be synthesized by standard processes known to the skilled worker from the corresponding benzyl halides, for example benzyl chlorides or bromides, of the formula III and from the corresponding amines R5-NH₂, where R1 to R5 are as defined above, and X is F, Cl, Br or I, in particular Cl or Br.

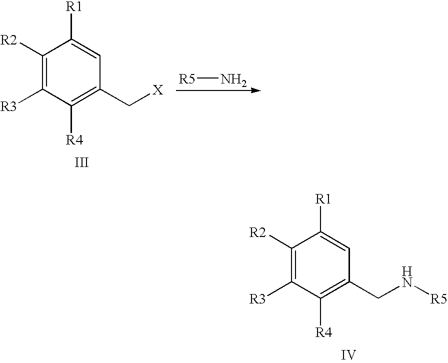

Alternatively, compounds of the formula IV can also be obtained by reductive amination of an aldehyde of the formula IIIa by standard processes known in the art, where R1 to R5 are as defined above.

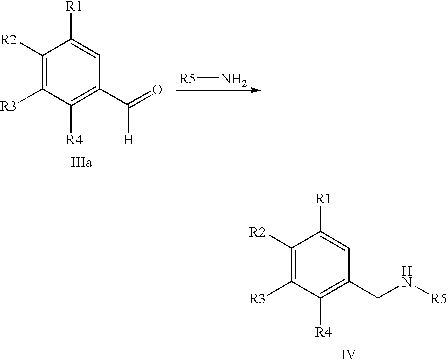

The compounds of the formulae III and IIIa, IX and R6-LG and R5-NH₂ can be obtained by purchase or can be prepared by or in analogy to processes described in the literature and well-known to one skilled in the art.

The preparation and purification of the products and/or intermediates takes place by conventional methods such as extraction, chromatography or crystallization and conventional dryings.

The invention relates to the administration of the compounds of formula I and the pharmaceutically acceptable salts thereof in the treatment of neurodegenerative disorders, memory impairments and other mental disorders such as dementia in the elderly, Alzheimer's, vascular dementias such as, for example, multi-infarct dementia, combinations of Alzheimer's and cerebrovascular disorders, tau mutations, prion diseases, polyglutamine expansion disorders such as, for example, Huntington's chorea and spinocerebellar ataxias, and Parkinsonism, and for improving memory. NHE-5 inhibitors are further suitable for the treatment of secondary dementias following and/or associated with infections such as, for example, with HIV, brain traumas, brain tumors or intoxications such as those due to drug or alcohol abuse.

The invention also relates to pharmaceutical compositions for human or veterinary use comprising an effective amount of a compound of the formula I and/or of a pharmaceutically acceptable salt thereof, as well as medicines for human or veterinary use comprising an effective amount of a compound of the formula I and/or of a pharmaceutically acceptable salt thereof alone or in combination with one or more other pharmacological active ingredients or medicaments.

Pharmaceutical compositions which comprise a compound of the formula I or its pharmaceutically acceptable salts can be administered orally, parenterally, intramuscularly, intravenously, rectally, nasally, by inhalation, subcutaneously or by a suitable transcutaneous dosage form, and the preferred administration depends on the particular manifestation of the disorder. The compounds of the formula I can moreover be used alone or together with pharmaceutical excipients, in particular both in veterinary and in human medicine. The medicaments comprise active ingredients of the formula I and/or their pharmaceutically acceptable salts generally in an amount of from 0.01 mg to 1 g per dose unit.

The pharmaceutical compositions of the present claimed invention may be formulated with excipients known in the pharmaceutical arts for administration, delivery and treatment. Besides solvents, gel formers, suppository bases, tablet excipients, and other active ingredient carriers, compositions may also comprise antioxidants, dispersants, emulsifiers, antifoams, masking flavors, preservatives, solubilizers or colorants.

For a form for oral use, the active compounds are mixed with the additives suitable for this purpose, such as carriers, stabilizers or inert diluents, and converted by conventional methods into suitable dosage forms such as tablets, coated tablets, two-piece capsules, aqueous, alcoholic or oily solutions. Examples of inert carriers which can be used are gum arabic, magnesia, magnesium carbonate, potassium phosphate, lactose, glucose or starch, especially corn starch. Preparation can take place both as dry and as wet granules. Examples of suitable oily carriers or solvents are vegetable or animal oils, such as sunflower oil or fish liver oil.

For subcutaneous, percutaneous or intravenous administration, the active compounds used are converted if desired with the substances customary for this purpose, such as solubilizers, emulsifiers or further excipients, into solution, suspension or emulsion. Examples of suitable solvents are: water, physiological saline solution or alcohols, e.g. ethanol, propanol, glycerol, as well as sugar solutions such as glucose or mannitol solutions, or else a mixture of the various solvents mentioned.

Suitable as pharmaceutical formulation for administration in the form of aerosols or sprays are, for example, solutions, suspensions or emulsions of the active ingredient of the formula I in a pharmaceutically acceptable solvent such as, in particular, ethanol or water, or a mixture of such solvents. The formulation may if required also comprise other pharmaceutical excipients such as surfactants, emulsifiers and stabilizers, and a propellant gas. Such a preparation normally comprises the active ingredient in a concentration of about 0.1 to 10, in particular of about 0.3 to 3, % by weight.

The dosage of the active ingredient of the formula I to be administered, and the frequency of administration, depend on the potency and duration of action of the compounds used; additionally also on the nature and severity of the disease to be treated, and on the gender, age, weight and individual response of the mammal to be treated.

On average, the daily dose of a compound of the formula I for a patient weighing about 75 kg is at least 0.001 mg/kg, preferably 0.1 mg/kg, to at most 30 mg/kg, preferably 1 mg/kg, of body weight, even higher doses may also be necessary in acute situations, for instance immediately after suffering apneic states at high altitude. Up to 300 mg/kg per day may be necessary especially on i.v. use, for instance for an infarct patient in an intensive care unit. The daily dose may be divided into one or more, for example, up to 4, single doses.

It will be appreciated that every suitable combination of the compounds of the invention with one or more of the aforementioned compounds and optionally one or more other pharmacologically active substances is regarded as falling within the protection conferred by the present invention. The examples detailed below are provided to better describe and more specifically set forth the compounds, processes and methods of the present invention. It is to be recognized that they are for illustrative purposes only however, and should not be interpreted as limiting the spirit and scope of the invention as later recited by the claims that follow. Moreover, in the experimental descriptions and examples below, a number of abbreviations are used therein which may be defined as follows:

### List of Abbreviations Used:

AMPA receptor-coupled channels which can be activated by α-amino-3-hydroxy-5-methyl isoxazole-4-propionate
CA 1 CA=cornu ammonis (Ammon's horn), CA region 1 in the hippocampus
EA ethyl acetate
EPSP excitatory post-synaptic potential
ES⁺ electron spray
HEP n-heptane
Conc. NH₃ saturated aqueous NH₃ solution
LTP long-term potentiation
LTP1 early LTP (phase of LTP)
MeOH methanol
mp melting point
MS mass spectroscopy
NMDA receptor-coupled channel which can be activated by N-methyl-D-aspartate
RT room temperature
STP short-term potentiation (phase of LTP)
THF tetrahydrofuran

### EXAMPLE 1

### Preparation of N-Diaminomethylene-4-(6,8-dichloro-2-methyl-1,2,3,4-tetrahydroisoquinolin-4-yl)benzenesulfonamide, dihydrochloride

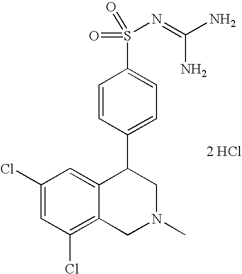

Guanidine (0.36 g) is suspended in 30 ml of anhydrous THF under argon, and 0.40 g of 4-(6,8-dichloro-2-methyl-1,2,3,4-tetrahydroisoquinolin-4-yl)benzenesulfonyl chloride (WO2003048129) is added. The mixture was stirred at RT for 24 h and then the THF was distilled off. 10 ml of water were added to the residue, and the precipitate was filtered off. It was washed with 10 ml of water and dried in vacuo. The solid was then suspended in 10 ml of EA, and 10 ml of a saturated solution of HCl in diethyl ether were added. The volatile constituents were removed in vacuo, and the residue was suspended in 10 ml of EA and stirred at RT for 5 h. The precipitate was then filtered off and dried in vacuo. 0.45 g was obtained, mp 140° C. (decomposition).

R_{f} (EA/HEP/CH₂Cl₂/MeOH/conc. NH₃=10:5:5:5:1)=0.30 MS (ES⁺): 412

In order to obtain pharmacological data for NHE-5 inhibition, the recovery in the intracellular pH (pHᵢ) of LAP1 cells, which stably express the different subtypes of the sodium-proton exchanger (NHE), was determined after an acidification. This recovery occurs even under bicarbonate-free conditions in the case of functioning NHE. To this end, the pHᵢ was determined with the pH-sensitive fluorescent dye BCECF (Molecular Probes, Eugene, Oreg., USA; the precursor BCECF-AM is used). The cells were first incubated with BCECF (5 μM BCECF-AM) in NH₄Cl buffer (NH₄Cl buffer: 115 mM choline Cl, 20 mM NH₄Cl, 5 mM KCl, 1 mM CaCl₂, 1 mM MgCl₂, 20 mM Hepes, 5 mM glucose; a pH of 7.4 is established with 1 M KOH). The intracellular acidification was induced by washing the cells incubated in NH₄Cl buffer with NH₄Cl-free buffer (133.8 mM choline chloride, 4.7 mM KCl, 1.25 mM CaCl₂, 1.25 mM MgCl₂, 0.97 mM K₂HPO₄, 0.23 mM KH₂PO₄, 5 mM Hepes, 5 mM glucose; a pH of 7.4 is established with 1 M KOH). After the washing operation, 90 μl of the NH₄Cl-free buffer were left on the cells. The pH recovery was started by the addition of 90 μl of Na⁺-containing buffer (133.8 mM NaCl, 4.7 mM KCl, 1.25 mM CaCl₂, 1.25 mM MgCl₂, 0.97 mM Na₂HPO₄, 0.23 mM NaH₂PO₄, 10 mM Hepes, 5 mM glucose; a pH of 7.4 is established with 1 M NaOH) in the analytical instrument (FLIPR, “Fluorometric Imaging Plate Reader”, Molecular Devices, Sunnyvale, Calif., USA). The BCECF fluorescence was determined at an excitation wavelength of 498 nm and the FLIPR emission filter 1 (band gap from 510 to 570 nm). The subsequent changes in fluorescence were registered for two minutes at NHE-5 as a measure of the pH recovery. For the calculation of the NHE-inhibitory potential of the tested substances, the cells were tested first in buffers in which full pH recovery, or none at all, took place. For full pH recovery (100%), the cells were incubated in Na⁺-containing buffer (see above), and Na⁺-free buffer for the determination of the 0% value (see above). The substances to be tested were made up in Na⁺-containing buffer. The recovery in the intracellular pH at each tested concentration of a substance was expressed in percent of the maximum recovery. From the percentages of the pH recovery, the IC₅₀ value of the particular substance for the individual NHE subtypes was calculated by means of the program XLFit (idbs, Surrey, UK).

| | NHE-5 IC₅₀ [μM] |
|---|---|
| Example 1 | 0.37 |

The LTP in the CA 1 region of the hippocampus section is the LTP which has been best characterized in vitro. The stratification and input structure permits field potential measurements over several hours. In the NHE studies, a weak tetanus based on the theta rhythm and which induces an early LTP returns to the initial value within three hours was used (Journal of Neuroscience, 18(16), 6071(1998); Eur J Pharmacol. 502: 99-104, 2004). It has recently been confirmed that an increasing number of theta burst trains induces an LTP of increasing magnitude and persistence (J Neurophysiol. 88:249-255, 2002), i.e. that a single weak stimulus induces an unsaturated LTP, not the maximally achievable saturated type of LTP. Both the magnitude (Behnisch, Reymann et al., Neurosci. Lett. 1998, 253(2): 91-94) and persistence (e.g. Neuropeptides 26: 421-427, 1994) of this LTP can be improved or adversely affected by substances. The early LTP which we generate in our investigations is likewise unsaturated. It is thus possible to ascertain a substance-induced improvement or deterioration in the early LTP. The early LTP investigated is composed of the STP component, which is known to persist for about 30 minutes (Nature 335: 820-824, 1988), and the LTP 1 component, which usually persists in the first 1-2 hours after LTP induction (Learn Mem. 3: 1-24, 1996).

The short (30-60 minute) recording of the initial values before the tetanus permits early effects of the substance to be investigated on normal, unstimulated synaptic transmission to be investigated. Since the principal excitatory synapses are glutamatergic (J Clin Neurophysiol. 9: 252-263, 1992), i.e. the monosynaptic field EPSP is determined very substantially by AMPA and only to a considerably smaller extent by NMDA receptors, an effect on glutamatergic transmission is thus simultaneously indirectly tested. To this end the hippocampus sections of the brain in Seven to eight (7-8) week old male Wistar rats were studied (in vitro) were sacrificed exposure cranium of brain was opened by dorsal to ventral cutting along the sagittal suture of the skull. The brain was incised between the hemispheres and, starting with the right hemisphere, the hippocampus was pulled out using a blunt implement.

The exposed hippocampus was transferred to a cooling block with moist filter paper, and the excess moisture was drawn off with the aid of another filter paper. This hippocampus fixed to the cooling block in this way was placed on the chopper and rotated horizontally until the hippocampus was at an appropriate angle to the cutting blade.

In order to maintain the laminar structure of the hippocampus it was necessary to cut the hippocampus at an angle of about 70 degrees in relation to the cutting blade (chopper).

The hippocampus was sliced at intervals of 400 μm. The sections were taken off the blade with the aid of a very soft, thoroughly wetted brush (marten hair) and transferred into a glass vessel with cooled nutrient solution gassed with 95% O₂/5% CO₂. The total duration of the preparation lasted no more than 5 min. The sections lay under a liquid level of 1-3 mm in a temperature-controlled chamber (33° C.). The flow rate was 2.5 ml/min. The pregassing took place under a slightly raised pressure (about 1 atm) and through a microneedle in the prechamber. The section chamber was connected to the prechamber so that it was possible to maintain a minicirculation. The minicirculation was driven by the 95% O₂/5% CO₂ flowing out through the microneedle. The freshly prepared hippocampus sections were adapted in the section chamber at 33° C. for at least 1 h.

A test stimulus level was determined (fEPSP) 30% of the maximum EPSP and measurement of the focal potentials were measured by a monopolar stimulation electrode that consisted of lacquered stainless steel and a constant-current, biphasic stimulus generator (WPI A 365) which were used for local stimulation of Schaffer collaterals (voltage: 1-5 V, pulse width of one polarity 0.1 ms, total pulse 0.2 ms).

Measurement: glass electrodes (borosilicate glass with filament, 1-5 MOhm, diameter: 1.5 mm, tip diameter: 3-20 μm) which were filled with normal nutrient solution were used to record the excitatory post-synaptic potentials (fEPSP) from the stratum radiatum. The field potentials were measured versus a chlorinated silver reference electrode located at the edge of the section chamber using a DC voltage amplifier. The field potentials were filtered through a low-pass filter (5 kHz).

The slope of the field potentials: fEPSPs (fEPSP slope) was determined for the statistical analysis of the experiments. The recording, analysis and control of the experiment took place with the aid of a software program (PWIN) which was developed in the department of neurophysiology. The formation of the average fEPSP slopes and the respective time points and construction of the diagrams took place with the aid of the Excel software, with automatic data recording by an appropriate macro.

### Nutrient Medium (Ringer's Solution):

| Substance | in mM | for 1 l in g |
|---|---|---|
| NaCl | 124 | 7.248 |
| KCl | 4.9 | 0.356 |
| MgSO4* 7H2O | 1.3 | 0.321 |
| CaCl2+ anhydrous | 2.5 | 0.368 |
| KH2PO4 | 1.2 | 0.164 |
| NaHCO3 | 25.6 | 2.152 |
| Glucose | 10 | 1.802 |
| Osmolarity in mOsm | 330 | |
| PH | 7.4 | |

The hippocampus from Example 1 was dissolved in DMSO and diluted with Ringer's solution to the final concentration for the experiments (final concentration 0.01% DMSO). In the control experiments, the baseline synapic transmission was initially recorded for 60-120 minutes. Subsequently, two double pulses were administered four times at an interval of 200 ms, with an interpulse interval of 10 ms for the double pulses and a width of 0.2 ms for the individual pulses (weak tetanus). The resulting potentiation of the EPSPs was recorded for at least 60 minutes.

In order to test the effect of the NHE-5 inhibitor, the baseline was again recorded initially for 60-120 minutes. The NHE-5 inhibitor (10 μM) was flushed for 20 minutes before the stimulation. Two double pulses were administered four times at an interval of 200 ms as in the control experiments, with an interpulse interval of 10 ms for the double pulses and a width of 0.2 ms for the individual pulses. The substance was washed out 20 minutes after stimulation, and the potentiation of the EPSP was recorded for at least 60 minutes.

### Result:

The compound of example 1 had no intrinsic effect on synaptic transmission in the concentration used.

The potentiation after administration of example 1 was still under 137% of the baseline 80 min after the stimulus, whereas the potentiation under control conditions had almost returned to the baseline level, at 113% of the baseline. This shows clearly that even 10 μM of the compound of example 1 improve maintenance of the weak LTP.

What is claimed is:

1. A method for the treatment of neurodegenerative disorders and memory impairments comprising the administration of a compound of formula I: 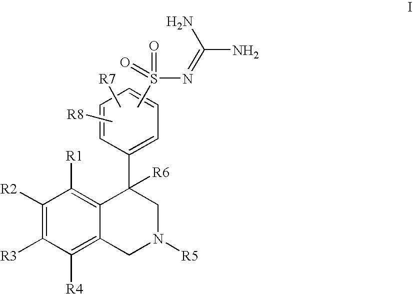
wherein:
R1, R2, R3 and R4 are independently selected from the group consisting of hydrogen, F, Cl, Br, I, CN, NO₂ and R11-(CₘH₂ₘ)-Aₙ;
m zero, 1, 2, 3 or 4;
n zero or 1;
R11 hydrogen, methyl or CₚF₂ₚ₊₁;
A oxygen, NH, N(CH₃) or S(O)_{q};
p 1, 2 or 3;
q zero, 1 or 2;
R5 is selected from the group consisting of hydrogen, alkyl having 1, 2, 3, 4, 5 or 6 Carbon atoms and cycloalkyl having 3, 4, 5 or 6 Carbon atoms;
R6 is selected from the group consisting of hydrogen, OH, F, CF₃, alkyl having 1, 2, 3 or 4 Carbon atoms and cycloalkyl having 3, 4, 5 or 6 Carbon atoms;
R7 and R8 are independently selected from the group consisting of hydrogen, F, Cl, Br, CN, CO₂R12, NR13R14 and R16-(CₘₘH₂ₘₘ)-Eₙₙ-;
R12 is selected from the group consisting of hydrogen, alkyl having 1, 2, 3 or 4 Carbon atoms and cycloalkyl having 3, 4, 5 or 6 Carbon atoms;
R13 and R14 are independently selected from the group consisting of hydrogen, alkyl having 1, 2, 3 or 4 Carbon atoms and cycloalkyl having 3, 4, 5 or 6 Carbon atoms;
or
R13 and R14 together with the nitrogen atom to which they are bonded, form a 4, 5, 6 or 7 membered ring in which one CH₂ group is optionally replaced by NR15, S or oxygen; and wherein
R15 is selected from the group consisting of hydrogen, alkyl having 1, 2, 3 or 4 Carbon atoms and cycloalkyl having 3, 4, 5 or 6 Carbon atoms;
mm is zero, 1, 2, 3 or 4;
nn is zero or 1;
R16 is hydrogen, methyl or CₚₚF₂ₚₚ₊₁;
E oxygen or S(O)_{qq}; wherein
pp is 1, 2 or 3;
qq is zero, 1 or 2;
or a pharmaceutically acceptable salt thereof.

2. The method as recited in claim 1 comprising the compound of formula I wherein: R1, R2, R3 and R4 are independently selected from the group consisting of hydrogen, F, Cl, Br, CN and R11-(CₘH₂ₘ)-Aₙ-;
wherein
m is zero or 1;
n is zero or 1;
R11 is hydrogen, methyl or CₚF₂ₚ₊₁;
A is oxygen, NCH₃ or S(O)_{q};
p is 1 or 2;
q is zero, 1 or 2;
R5 is selected from the group consisting of hydrogen, methyl, ethyl and cyclopropyl;
R6 is hydrogen or methyl;
R7 and R8 are independently selected from the group consisting of hydrogen, F, Cl, CN, CO₂R12, NR13R14 and R16-(CₘₘH₂ₘₘ)-Eₙₙ-;
R12 is selected from the group consisting of hydrogen, methyl and ethyl;
R13 and R14 are independently selected from the group consisting of hydrogen, alkyl having 1, 2, 3 or 4 Carbon atoms and cycloalkyl having 3, 4, 5 or 6 Carbon atoms;
or
R13 and R14, together with the nitrogen atom to which they are bonded, form a 5, 6 or 7 membered ring in which one CH₂ group is optionally replaced by NR15, S or oxygen;
R15 hydrogen, alkyl having 1, 2, 3 or 4 Carbon atoms or cycloalkyl having 3, 4, 5 or 6 Carbon atoms;
mm zero, 1 or 2;
nn zero or 1;
R16 hydrogen, methyl or CₚₚF₂ₚₚ₊₁;
E oxygen or S(O)_{q}q;
pp 1 or 2;
qq zero, 1 or 2;
or a pharmaceutically acceptable salt thereof.

3. The method as recited claim 2 comprising the compound of formula I wherein:
R1 and R3 are both hydrogen;
R2 and R4 are independently selected from the group consisting of hydrogen, F, Cl, NH₂, NHCH₃ and N(CH₃)₂;
R5 is hydrogen, methyl, ethyl or cyclopropyl;
R6 is hydrogen or methyl;
R7 and R8 are both hydrogen;
or a pharmaceutically acceptable salt thereof.

4. The method as recited in claim 3, wherein the compound of formula 1 is N-diaminomethylene-4-(6,8-dichloro-2-methyl-1,2,3,4-tetrahydroisoquinolin-4-yl)benzenesulfonamide or a pharmaceutically acceptable salt thereof.

5. The method of claim 4 wherein the compound of formula 1 is useful in the preparation of a pharmaceutical composition for the treatment of dementing disorders.

6. The method of claim 5 wherein the compound of formula 1 is useful in the preparation of a pharmaceutical composition for the treatment of dementia in the elderly.

7. The method of claim 6 wherein the compound of formula 1 is useful in the preparation of a pharmaceutical composition for the treatment of Alzheimer's disease vascular dementias, combinations of Alzheimer's and cerebrovascular disorders, tau mutations, prion disorders, polyglutamine expansion disorders and Parkinsonism.

8. A pharmaceutical composition comprised of the compound of formula 1: 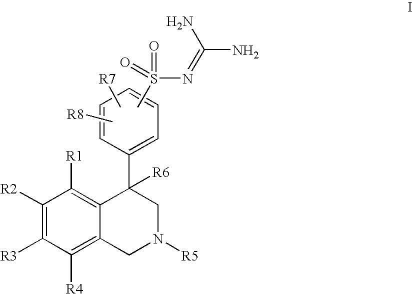
wherein:
R1, R2, R3 and R4 are independently selected from the group consisting of hydrogen, F, Cl, Br, CN and R11-(CₘH₂ₘ)-Aₙ-;
wherein
m is zero or 1;
n is zero or 1;
R11 is hydrogen, methyl or CₚF₂ₚ₊₁;
A is oxygen, NCH₃ or S(O)_{q};
p is 1 or 2;
q is zero, 1 or 2;
R5 is selected from the group consisting of hydrogen, methyl, ethyl and cyclopropyl;
R6 is hydrogen or methyl;
R7 and R8 are independently selected from the group consisting of hydrogen, F, Cl, CN, CO₂R12, NR13R14 and R16-(CₘₘH₂ₘₘ)-Eₙₙ-;
R12 is selected from the group consisting of hydrogen, methyl and ethyl;
R13 and R14 are independently selected from the group consisting of hydrogen, alkyl having 1, 2, 3 or 4 Carbon atoms and cycloalkyl having 3, 4, 5 or 6 Carbon atoms;
or
R13 and R14, together with the nitrogen atom to which they are bonded, form a 5, 6 or 7 membered ring in which one CH₂ group is optionally replaced by NR15, S or oxygen;
R15 hydrogen, alkyl having 1, 2, 3 or 4 Carbon atoms and cycloalkyl having 3, 4, 5 or 6 Carbon atoms;
mm zero, 1 or 2;
nn zero or 1;
R16 hydrogen, methyl or CₚₚF₂ₚₚ₊₁;
E oxygen or S(O)_{q}q;
pp 1 or 2;
qq zero, 1 or 2;
or a pharmaceutically acceptable salt thereof.

9. The pharmaceutical composition of claim 8 comprising formula I wherein:
R1 and R3 are both hydrogen;
R2 and R4 are independently selected from the group consisting of hydrogen, F, Cl, NH₂, NHCH₃ and N(CH₃)₂;
R5 is hydrogen, methyl, ethyl or cyclopropyl;
R6 is hydrogen or methyl;
R7 and R8 are both hydrogen;
or a pharmaceutically acceptable salt thereof.

10. The pharmaceutical composition of claim 9 comprising formula I for the treatment of secondary dementias following and/or associated with infections, brain traumas, brain tumors or intoxications.

11. The pharmaceutical composition of claim 9 comprising formula I for the treatment of improving memory.
